(19) United States
(12) Patent Application Publication
(10) Pub. No.: US 2015/0374861 A1
(43) Pub. Date: Dec. 31, 2015

(54) KIT FOR TUMOR IMAGING

(71) Applicant: THOMAS JEFFERSON UNVERSITY, Philadelpha, PA (US)

(72) Inventor: Mathew L. Thakur, Cherry Hill, NJ (US)

(21) Appl. No.: 14/767,927

(22) PCT Filed: Feb. 11, 2014
(86) PCT No.: PCT/US14/15752

§ 371 Date: Aug. 14, 2015

Related U.S. Application Data
(60) Provisional application No. 61/765,312, filed on Feb. 15, 2013.

(51) Int. Cl. A61K 51/08; A61K 51/04

(52) U.S. Cl. CPC A61K 51/08; A61K 51/0482; A61K 51/0478

(57) ABSTRACT

This invention relates to kits for tumor imaging and methods for preparing the kits using a composition having the structure— His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-yAba-Lys (R) (SEQ ID NO:1), wherein the y-amino group of the lysine residue is coupled to a chelator disclosed in this application.

### INTRODUCTION

This application claims the benefit of priority of U.S. Provisional Application No. 61/765,312, filed Feb. 15, 2013, the content of which is incorporated herein by reference in its entirety.

This invention was made with government support under contract number CA 109231 awarded by the National Institutes of Health. The government has certain rights in the invention.

### BACKGROUND

Of the approximately 1.6 million breast biopsies which were performed in the United States in 2011, about 288,130 breast cancers (BC) were diagnosed (230,480 invasive and 57,650 in situ) (DeSantis, et al. (2011) CA Cancer J. Clin. 61:409-418; Elter, et al. (2011) Med. Phys. 2007, 34(11):4164-4172), but over 1.3 million of these biopsies resulted in a benign diagnosis. While it is critical to diagnose these cancers, the high number of benign biopsies that are performed in this process creates significant patient morbidity and potentially unnecessary health care costs. There are continued advances in imaging modalities to detect BC, including digital mammography, MRI, CT, US, F-18-FDG and Tc-99m sestamibi, however all of these modalities have limited specificity and all continue to produce many false positive and false negative examinations (Uematsu, et al. (2002) Breast Cancer 9:62-68; Berg, et al. (2004) Radiology 233:830-849; Ruibal, et al. (2008) Med. Clin. 130(9):332-333; Yang & Tse (2004) AJR Am. J. Roentgenol. 182:101-110; Elmore, et al. (2005) JAMA 293:1245-1256; Ghai, et al. (2005) AJR Am. J. Roentgenol. 185:481-487; Chagpar, et al. (2006) Ann. Surg. 243:257-264; Xu, et al. (2011) Nucl. Med. Comm. 32:980-988; Xue, et al. (2012) Eur. J. Surg. Oncol. 38(5):375-381). Given the cost, unnecessary benign biopsies represent a serious health care burden. There is a compelling need for an innovative approach that would decrease the number of unnecessary benign biopsies while still detecting malignancies.

Recent approaches to drug discovery focus on understanding the genesis of disease and the biomedical pathways controlling disease at a molecular level. Previous studies have demonstrated that VPAC1 receptors (combined for vasoactive intestinal and pituitary adenylate cyclase activating peptide) are overexpressed in high density on BC cells (Reubi, et al. (2000) Cancer Res. 60(11):3105-3112). VPAC1 receptors encode a G protein involved in cell proliferation, cell differentiation, as well as in survival of BC cells. On stroma, normal cells, and benign masses only a few VPAC1 receptors are expressed (Reubi, et al. (2000) supra; Zia, et al. (1996) Cancer Res. 56(15):3486-89; Leyton, et al. (1999) Breast Canc. Res. Treat. 56(2):177-186; Moody & Gozes I (2007) Curr. Pharm. Des. 13(11):1099-1104; Valdehita, et al. (2010) Peptides 31(11):2035-2045; Valdehita, et al. (2012) Mol. Cell Endocrinol. 348(1):241-246).

Radiolabeled biomolecules with high affinities for VPAC1 receptors have been developed and analyzed in a preclinical setting (Chakder & Rattan (1993) J. Pharm. Expt. Therapeut. 266:392-399; Thakur, et al. (2000) J. Nucl. Med. 41:107-110; Pallela, et al. (1999) J. Nucl. Med. 40(2):352-360; Kolan, et al. (1997) J. Label. Comp. Radiopharmaceut. 40:455-457; Thakur, et al. (2004) J. Nucl. Med. 45:1381-1389; Zhang, et al. (2007) Reg. Peptides 144:91-100; Thakur (2009) Semin. Nucl. Med. 39:236-246; Thakur, et al. (2010) J. Nucl. Med. 51:106-111; Zhang, et al. (2008) J. Nucl. Med. 49:112-121; U.S. Pat. No. 6,855,308). Based on their high affinity for VPAC1 receptors, peptide constructs, modified by radiolabeling with Tc99m (t½—6 hours, γ—140 keV), were generated and evaluated for receptor affinity (Kd), receptor specificity, in vivo stability and tissue distribution (Thakur, et al. (2000) supra; Pallela, et al. (1999) supra; Kolan, et al. (1997) supra; Thakur, et al. (2004) supra; Zhang, et al. (2007) supra; Thakur (2009) supra; Thakur, et al. (2010) supra; Zhang, et al. (2008) supra). In addition, peptides were labeled with β+(19%, 656 keV) emitting Cu-64 (t½—12.8 hours) for positron emission tomography (PET) with N₂S₂ as a chelating agent. Kd values, tissue distribution studies in athymic nude mice bearing T47D human BC, receptor blocking studies, receptor affinity, and in vivo stability were examined (Thakur, et al. (2004) supra; Zhang, et al. (2007) supra; Thakur (2009) supra; Thakur, et al. (2010) supra; Zhang, et al. (2008) supra). Of the compounds produced, Cu-64-TP3805 not only imaged all xenografted human BC in athymic nude mice (tumor uptake 6.35±1.28% ID/g at 24 hours post-injection), this compound also localized all (n=8), spontaneously grown BC (visible 5, invisible 1 and metastatic 2) lesions in transgenic MMTVneu mice (n=9) (Thakur, et al. (2010) supra). Furthermore, the Cu-64-TP3805 PET images were normal for two lesions that had negligible expression of VPAC1 receptors. The eight malignant lesions were confirmed by histology and expressed VPAC1 receptors as determined by RT-PCR (Thakur, et al. (2010) supra). Based upon this analysis, this compound is of use in diagnosing breast cancer.

### SUMMARY OF THE INVENTION

The present invention provides kits for tumor imaging. In one embodiment, the kit of this invention is composed of a lyophilized composition including glucoheptonate, glycine buffer, and a compound having the structure His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-γAba-Lys(R) (SEQ ID NO:1), wherein R is a chelator coupled to the γ-amino group of the lysine residue and said chelator is:

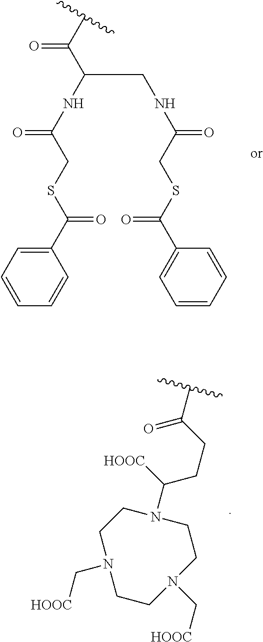 [SMILES: *C(=O)C(CNC(=O)CSC(=O)c1ccccc1)NC(=O)CSC(=O)c1ccccc1 ; *C(=O)CCC(C(=O)O)N1CCN(CC(=O)O)CCN(CC(=O)O)CC1]

In some embodiments, the kit is stored at 4° C. In other embodiments, the kit is stored at −20° C. In yet further embodiments, the kit includes 20-250 μg of the compound, 50 to 500 μg of glucoheptonate, and optionally ⁶⁴Cu, ⁶⁸Ga, ⁸⁹Zr or ^{99m}TC. A method for preparing the kit is also provided.

In another embodiment, the kit of the invention is composed of a compound having the structure His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-γAba-Lys(R) (SEQ ID NO:1), wherein R is a chelator coupled to the γ-amino group of the lysine residue and wherein said compound is immobilized on a substrate.

### DETAILED DESCRIPTION OF THE INVENTION

It has now been shown that Cu-64-TP3805 can image primary tumors in human breast cancer patients. Of the twenty tumors analyzed, all lesions were malignant. In addition, in whole body positron emission tomography (PET)/computed tomography (CT) imaging, four involved sentinel lymph nodes (two in one patient and one each in the other two patients) and were delineated clearly. In this study, two other observations were made that were noteworthy. First, the Cu-64-TP3805 uptake was rapid, i.e., minutes post-injection. Therefore, a 68 minute half-lived, generator-produced Ga-68 can also be used. The positron emission of Ga-68 is 88%, more than four times greater than that of Cu-64. This permits administration of less than 150 MBq (˜4 mCi) of the Ga-68 without compromising the image quality, yet significantly reducing the radiation burden to the subjects. In either case, images can be obtained 15 minutes post-injection without requiring patient fasting or monitoring of blood sugar level.

Second, it was observed that a Positron Emission Mammography (PEM) uptake value of 15 minutes post-injection did not alter up to 5 hours after imaging. This indicated that Cu-67-TP3805 could be used as a therapeutic agent without altering its chemistry or preparation procedure. Copper-67 is a β⁻ (100%) emitter, with a half-life of 2.44 days and is considered as a radionuclide of therapeutic importance (Knogler, et al. (2007) Clin. Cancer Res. 13:603-611).

Accordingly, the present invention provides kits for tumor imaging and methods for preparing such kits. In accordance with this invention, a kit for tumor imaging, in particular breast cancer tumor imaging, includes a composition composed of a mixture of glucoheptonate, glycine buffer, and an imaging compound having the structure His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-γAba-Lys(R) (SEQ ID NO:1), wherein R is a chelator coupled to the γ-amino group of the lysine residue and said chelator is:

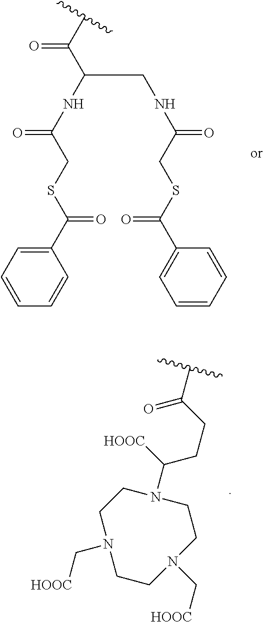 [SMILES: *C(=O)C(CNC(=O)CSC(=O)c1ccccc1)NC(=O)CSC(=O)c1ccccc1 ; *C(=O)CCC(C(=O)O)N1CCN(CC(=O)O)CCN(CC(=O)O)CC1]

As an alternative, other suitable chelating agents for use in the present invention include linear, cyclic and branched polyamino-polycarboxylic acids and their phosphorous oxyacid equivalents, for example ethylenediamine-N,N,N′,N′-tetraacetic acid (EDTA); N,N,N′,N″,N″-diethylene-triaminepentaacetic acid (DTPA); 1,4,7,10-tetraazocyclododecane-N,N′N″,N″-tetraacetic acid (DOTA); 1,4,7,10-tetraazo-cyclododecane-N,N′N″-triacetic acid (DO3A); 1-oxa-4,7,10-triazacyclododecane-N,N′N″-triacetic acid (OTTA); trans(1,2)-cyclohexanodiethylene-triamine-pentaacetic acid (CDTPA); 1-oxa-4,7,10-triazacyclododecantriaacetic acid (DOXA); 1,4,7-triazacyclononanetriacetic acid (NOTA); and 1,4,8,11-tetraazacyclotetradecanetetraacetic acid (TETA).

The glucoheptonate and glycine buffer can each be obtained from commercial sources and used without additional purification. In some embodiments, the kit further includes SnCl₂.2H₂O, e.g., between 20 and 1000 μg of SnCl₂.2H₂O. In other embodiments, the kit includes between and 500 μg of SnCl₂.2H₂O or between 50 and 200 μg of SnCl₂.2H₂O. In particular embodiments, the kit includes 100 μg of SnCl₂.2H₂O. Similarly, in some embodiments the kit includes between 10 and 500 μg of glucoheptonate, between 50 and 500 μg of glucoheptonate, or more particularly, 100 μg glucoheptonate. The glycine buffer can be prepared using conventional methods and preferably has a pH in the range of 5 to 9, more preferably, 6 to 9, or most preferably 7 to 9.

The imaging compound can be prepared as described herein using conventional chemical synthesis methods. In some embodiments, the kit contains between 5 and 500 μg of the imaging compound. In other embodiments, the kit contains between 10 and 100 μg of the imaging compound. In particular embodiments, the kit contains 20 and 250 μg of the imaging compound.

Desirably, the components of the composition of the kit are sterile, combined under aseptic conditions, and provided in a container (e.g., a septum-sealed vial).

In particular embodiments, the kit also includes a radionuclide, e.g., a positron emitting radionuclide. In one embodiment, the radionuclide is ⁶⁴Cu. In another embodiment, the radionuclide is ⁶⁸Ga. In other embodiments, the radionuclide is ⁸⁹Zr or ^{99m}Tc.

In addition to the above-referenced components, the kit may optionally further include additional components such as a radioprotectant, antimicrobial preservative, pH-adjusting agent or filler. By the term “radioprotectant” is meant a compound which inhibits degradation reactions, such as redox processes, by trapping highly-reactive free radicals, such as oxygen-containing free radicals arising from the radiolysis of water. By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the imaging composition post-reconstitution, i.e., in the imaging product itself. The antimicrobial preservative may, however, also optionally be used to inhibit the growth of potentially harmful micro-organisms in one or more components of the kit of the present invention prior to reconstitution. Suitable antimicrobial preservatives include parabens (e.g., methyl, ethyl, propyl or butyl paraben or mixtures thereof); benzyl alcohol; phenol; cresol; and cetrimide. By the term “filler” is meant a bulking agent which may facilitate material handling during production and lyophilization. Suitable fillers include inorganic salts such as sodium chloride, and water soluble sugars or sugar alcohols such as sucrose, maltose, mannitol or trehalose.

In an alternative embodiment of this invention, the kit contains an imaging compound, as described herein, adhered, affixed or immobilized to a substrate, e.g., a Petri dish, test well, microtiter plate, test tube, sample pad, test strip, bead or the like. Natural, synthetic, or naturally occurring materials that are synthetically modified, can be used as the substrate including, but not limited to, cellulose materials such as paper, cellulose, and cellulose derivatives such as cellulose acetate and nitrocellulose; fiberglass; cloth, both naturally occurring (e.g., cotton) and synthetic (e.g., nylon); porous gels such as silica gel, agarose, dextran, and gelatin; porous fibrous matrixes; starch-based materials, such as SEPHADEX brand cross-linked dextran chains; ceramic materials; films of polyvinyl chloride and combinations of polyvinyl chloride-silica; beads composed of polystyrene, polymethylacrylate, polyacrylamide, polypropylene, latex, polytetrafluoroethylene, polyacrylonitrile, polycarbonate, glass or similar materials; and the like. Other useful substrates are magnetic or paramagnetic particles.

The substrate of the instant kit can be derivatized to contain chemically active groups that can be coupled to the imaging compounds by simple chemical reactions, e.g., via a conventional disulphide, thioether or thiol-maleimide linkage or via amide linkage through the C-terminal carboxylic acid.

The substrate should not interfere with the production of a detectable signal and be compatible with biological samples being assayed, e.g., blood, plasma, urine, sputum, vaginal fluid, aspirated tissue samples and the like. The substrate should have a reasonable inherent strength, or strength can be provided by means of a supplemental support.

The particular dimensions of the substrate will be a matter of convenience, depending upon the size of the test sample involved, the assay protocol, the means for detecting and measuring the signal, and the like.

The kits of the invention can further include instructions for one or more of; reconstituting the composition containing the imaging agent, radiolabeling the imaging compound with a radionuclide, administering the composition to a subject, and interpreting results. Moreover, the kit can include photographic examples of labeling of tissues with and without cancer.

A kit of the invention can be prepared, e.g., as exemplified herein, by combining, in a container, glucoheptonate, glycine buffer, and an imaging compound; freezing the combination of reagents; lyophilizing the frozen combination, introducing sterile nitrogen gas into the container, sealing the container, and storing the container at a temperature of 4° C. or less. In one embodiment, the container is stored at 4° C. In another embodiment, the container is stored at −20° C.

The invention will be further described in the following examples, which do not limit the scope of the invention described in the claims.

### Example 1

### Materials and Methods

TP3805 Synthesis and Kit Preparation.

Briefly, the PACAP analog with a C-terminal diaminodithiol (N₂S₂) chelator was synthesized (Thakur (2009) supra; Thakur, et al. (2010) supra; Zhang, et al. (2008) supra; Anderson, et al. (2001) J. Nucl. Med. 42(2):213-221; Lewis, et al. (2001) Proc. Natl. Acad. Sci. 98(3):1206-1211) on a Wang resin using an ABI 341A peptide synthesizer (Applied Biosystems, Foster City, Calif.). Fmoc-Lys (ivDde) was first introduced at the C-terminus of the peptide, followed by 4-aminobutyric acid (γ-Aba). The 27-amino acid long PACAP sequence was then assembled by standard Fmoc coupling with the final histidyl residue being a t-Boc protected His(Trt) derivative. The capping t-Boc function was necessary to ensure that the N-terminal amino group remained protected during subsequent deprotection and coupling cycles performed at the γ-amino group of the C-terminal lysine. The ivDde group at the C-terminal lysine was then selectively removed with 2% hydrazine, followed by the successive additions of di-Fmoc-L-diaminopropionic acid, and S-benzoylthioglycolic acid. The resulting protected diaminedithiol (NS-benzoyl)₂-containing PACAP peptide was cleaved from the resin using trifluoroacetic acid (TFA) water:phenol:thioanisole/ethanedithiol (82.5:5:5:5:2.5) and precipitated with diethyl ether.

The crude peptide was purified to homogeneity by reverse-phase high pressure liquid chromatography (HPLC) (Waters, Milford, Mass.) on a VYDAC C4 column (5 μm, 10 mm×250 mm). The mass of the analog-chelator construct was confirmed by electrospray mass spectrometry. Following the general synthetic scheme, TP3805 was prepared, purified and characterized by American Peptide Company, Sunnyvale, Calif.

Kits were prepared aseptically in a laminar flow hood. All reagents were sterilized including 10 ml glass vials, rubber caps and aluminum sealing caps. All reagents were analytical grade obtained from Fisher Scientific, Inc. (Fair Lawn, N.J.) and used without further purification. The reagents added were 100 μg SnCl₂.2H₂O (10 mg/ml, 0.05 M HCl) containing 100 μg glucoheptonate (50 mg/ml, H₂O), 20 μg of TP3805 (10 mg/ml, and 0.1 M Na-acetate buffer, pH-5) and 200 μl of 0.2 M glycine buffer pH-9. The mixture was quickly frozen by placing the vials in an acetone/dry ice bath. The vials were then lyophilized for four hours, (Genevac SF50; Genevac, Berkshire, England). After lyophilization, sterile N₂ gas was introduced in the chamber, and the vials were sealed, labeled and stored at 4° C. until use. Stability of the kits was checked by HPLC and for the ability to be labeled with Cu-64.

Preparation of Cu-64-TP3805.

On the day of preparation, a kit vial was removed and brought to room temperature. The required quality of Cu-64 solution (Washington University, St. Louis, Mo.) was added to the vial (usually 6 mCi in <20 μl of 0.1 M HCl), followed by 200 μl of sterile water. The vial was then incubated at 50° C. for ninety minutes. The solution was then diluted by the addition of 2 ml sterile 0.9% NaCl.

The reaction mixture was analyzed by HPLC, with reverse-phase microbond column (Varian, Inc.) eluted with a linear, 28 minute gradient from 10% acetonitrile in 0.1% aqueous TFA to 90% acetonitrile in aqueous 0.1% TFA. Labeling efficiency of 95% or greater was considered as the criterion for kit stability. This rendered the specific activity of Cu-64-TP3805 to be 44.4 GBq (1.2 Ci)/μmol.

Sterility Test.

From the vial, a required activity for patient injection was drawn into a sterile syringe and measured in a calibrated ionization chamber CRC-15R, (Capintec Inc., Ramsey, N.J.). Approximately 100 μl of the solution was added to 10 ml tryptic soy broth (TSB) and incubated for seven days in a humidified 5% CO₂ incubator at 37° C. The test tube was observed daily for seven days to detect turbidity or microbial growth.

Patient Inclusion, Dose and Imaging.

Caucasian, Hispanic, African American or Asian non-pregnant women, eighteen years of age or older, with newly diagnosed, histologically proven BC were enrolled. Image guided percutaneous biopsy was performed two to six weeks prior to this imaging procedure. Each patient signed the informed consent form. For this feasibility study, a biostatistician determined that six patients be studied with PET and thirteen with PEM. This determination was based on the assumption that Cu-64-TP3805 will detect 80% of the lesions and there will be 87% power to conclude the use of this agent to diagnosis BC.

Each patient fasted for six hours prior to F-18-FDG injection. Before F-18-FDG injection, blood sugar was monitored. Patients with sugar levels below 250 mg/DL were enrolled. Each patient then received 370 MBq (10 mCi) of F-18-FDG through an indwelling intravenous catheter and one hour later underwent a PET or PEM scan. PET/CT images were obtained in supine position with two minute bed time using a Biograph-6 PET/CT scanner (Siemens, Inc. Knoxville, Tenn.).

For Cu-64-TP3805 imaging, patients neither fasted nor were blood sugar levels determined. Two patients received 111±10% MBq (3±10% mCi) Cu-64-TP3805, two patients received 127.5±10% MBq (3.5 mCi±10% MBq), and the two remaining patients received 148±10% MBq (4±10% mCi), wherein Cu-64-TP3805 was injected intravenously two to thirty days after the F-18-FDG scan. For Cu-64-TP3805 whole body scans, bed time was four minutes and images were obtained at 2 hours and 4 hours post-injection.

For PEM imaging, each patient received 148±10% MBq (4±10% mCi) Cu-64-TP3805 intravenously through an indwelling intravenous catheter. Images were obtained for both breasts in MLO and CC positions for 10 minutes per view. Data were collected at 15 minutes, 1 hour, 2 hours and 4 hours to 5 hours post-injection. Vital signs for each of the PET/CT and PEM patients were monitored prior to injection and then for every 30 minutes until 4 hours post-injection. At the end of the injection, the syringe and the tubing were flushed with 5 ml 0.9% NaCl and then the radioactivity remaining was measured. During the course of the PEM study, patients were allowed to drink or eat if they wished.

Image Analysis.

All images were read by two board certified nuclear medicine physicians and a board certified breast imaging physician. Image analysis was performed by a nuclear medicine fellow. For six patients with PET/CT, standardized uptake values (SUV) were calculated for the primary tumor site and metastatic lymph nodes, and compared for the respective radiotracers. Metabolic tumor volume was calculated using 50% isocontour geometry method for comparison, as it is relatively a better parameter, as compared to SUV.

For the thirteen patients with PEM scans, PEM uptake values (PUV) and metabolic tumor volumes were calculated for comparison. PEM uptake value/background intensity (PUV/BGV) ratios were also calculated with each radiotracer and compared for evaluation.

### Example 2

### Results

Cu-64-TP3805, Radiochemical Purity and Sterility.

For all Cu-64-TP3805 preparations, the radiochemical purity was determined by HPLC and averaged 97±2%. No further radiochemical purification was performed. Specific activity of the preparations averaged 44.4 GBq (1.2 Ci)/μmol. All preparations were sterile. Total radioactivity remaining in the syringe and the intravenous line was less than 5.5 MBq (150 μCi).

Patient Population.

All patients were recruited in a consecutive order as they consented. None of the patients had received any form of therapy for BC. The average age of the six PET/CT patients was 48.7±6.2 years (range 42-59 years). The average age of the thirteen PEM patients was 54±14.2 years (range 26-80 years). Out of the total of nineteen patients, two patients experienced a flushing sensation that resolved without any medication within minutes. The demographic patient data are given in Table 1 and critical results for each patient is in Table 2.

TABLE 1

| | | | | MBq Injected | |
|---|---|---|---|---|---|
| Patient | Patient | | | | Cu-64- |
| Number | Age | BC Type | ER/PR/HER2 | F-18-FDG | TP3805 |

PET/CT Patients

| | | | | | |
|---|---|---|---|---|---|
| 1 | 42 | IDC | +/+/− | 370 | 140.6 |
| 2 | 46 | IDC | +/+/− | 370 | 107.3 |
| 3 | 49 | IDC | +/+/− | 370 | 125.8 |
| 4 | 52 | IDC | −/−/+ | 370 | 125.8 |
| 5 | 44 | IDC | +/+/+ | 370 | 144.3 |
| 6 | 59 | IDC | +/+/− | 370 | 140.6 |

PEM Patients

| | | | | | |
|---|---|---|---|---|---|
| 1 | 68 | IDC | +/+/? | 370 | 144.6 |
| 2 | 57 | IDC | +/−/+ | 370 | 140.6 |
| 3 | 80 | IDC | +/+/− | 370 | 144.6 |
| 4 | 46 | ILoC | +/+/− | 370 | 142.5 |
| 5 | 39 | IDC | +/−/? | 370 | 159.1 |
| 6 | 59 | IDC | +/+/? | 370 | 162.8 |
| 7 | 39 | IDC | +/+/− | 370 | 144.3 |
| 8 | 55 | ILoC | +/+/− | 370 | 151.7 |
| 9 | 52 | IPaC | +/−/+ | 370 | 159.1 |
| 10 | 58 | IDC | +/+/− | 370 | 140.6 |
| 11 | 55 | IDC | +/−/? | 370 | 144.3 |
| 12 | 68 | IDC | +/+/− | 370 | 155.4 |
| 13 | 26 | HGMC | +/−/− | 370 | 151.7 |

IDC, Invasive Ductal Carcinoma; ILoC, Invasive Lobular Carcinoma; IPaC, Invasive Papilary Carcinoma; HGMC, High Grade Mammory Carcinoma.

TABLE 2

| Patient | SUV/PUV/BGV | | Tumor Volume (mm³) | |
|---|---|---|---|---|
| Number | F-18-FDG | Cu-64-TP3805 | F-18-FDG | Cu-64-TP3805 |

PET/CT Patients

| | | | | |
|---|---|---|---|---|
| 1 | 12.8 | 6.8 | 6084 | 5323 |
| | LN-1: 11.0 | LN-1: 4.9 | LN-1: 28 | LN-1: 28 |
| 2 | 5.5 | 7.0 | 5028 | 4714 |
| | LN-1: 1.8 | LN-1: 2.6 | LN-1: 59 | LN-1: 49 |
| | LN-2: 2.3 | LN-2: 2.4 | LN-2: 38 | LN-2: 31 |
| 3 | 2.2 | 2.1 | 113 | 113 |
| 4 | 5.2 | 4.7 | 2614 | 1726 |
| | LN-1: 5.2 | LN-1: 4 | LN-1: 509 | LN-1: 402 |
| 5 | 1.75 | 1.9 | 1385 | 1018 |
| 6 | 5.0 | 4.4 | 2617 | 2651 |

PEM Patients

| | | | | |
|---|---|---|---|---|
| 1 | 6.7 | 6.2 | 509 | 402 |
| 2 | 9.6 | 7.9 | 3818 | 4714 |
| 3 | 1.9 | 3.0 | 141 | 98 |
| 4 | 4.3 | 3.7 | 509 | 346 |
| 5 | 5.1 | 4.7 | 785 | 1357 |
| 6 | 11.6 | 11.8 | 7812 | 6912 |
| 7 | 2.9 | 2.2 | 3178 | 2828 |
| 8 | 3.0 | 3.3 | 445 | 549 |
| 9 | 7.3 | 5.8 | 1847 | 1357 |
| 10 | 6.2 | 5.4 | 1436 | 1767 |
| 11 | 2.6 | 3.1 | 268 | 381 |
| 12 | 2.7 | 3.1 | 1150 | 1767 |
| 13 | 4.5 | 2.7 | 696 | 1150 |

BGV, background value; PUV, PEM uptake value; SUV, standardized uptake value; LN, Lymph node.

Image Analysis.

In the whole body PET/CT group, all six patients had histologically proven invasive ductal carcinoma (IDC). Five of these were ER+, one ER−, five PR+, one PR−, and two HER2+ and four HER2−. The image quality for each of these patients, irrespective of the quantity of Cu-64-TP3805 they received, was excellent. Within these patients, there were a total of ten lesions detected both by F-18-FDG and Cu-64-TP3805. Out of these ten lesions, six were primary (one lesion in each patient) and four involved lymph nodes (two in one patient, and one each in the other two).

In the PEM group of thirteen patients, ten patients had histologically proven IDC, two had invasive lobular carcinoma (ILoC) and one had invasive papillary carcinoma (IPaC). Of these, all thirteen were ER+ and none were ER−. Eight patients had PR+ lesions and five had PR−. Of these two were HER2+, seven HER2−, and for four, HER2 status was indeterminate. One patient with ILoC had two distinct lesions (Table 1).

As shown in Table 2, the primary tumor volume in these six patents as determined by F-18-FDG scan ranged from 113 mm³ to 6084 mm³. The corresponding tumor volume range, as determined by the Cu-64-TP3805 scans, ranged from 113 mm³ to 5323 mm³. The Cu-64-TP3805 tumor volume was 90.6±16.1% of that of F-18-FDG. The F-18FDG lymph node volume range for the four nodes was 28 mm³ to 509 mm³ and for that of Cu-64-TP3805 was 28 mm³ to 402 mm³. The node volume as determined by Cu-64-TP3805 was 86.2±9.2% of that found by the F-18-FDG scan.

All six primary lesions and four malignant lymph nodes were unequivocally detected by Cu-64-TP3805. The F-18-FDG SUV (max) values for six primary lesions ranged from 1.75 to 12.8, and for the malignant lymph nodes 1.8 through 11.0. The corresponding Cu-64-TP3805 values were 1.9 through 11.8 for the primary lesions and 2.4 to 4.9 for the lymph nodes. The Cu-64-TP3805 SUV (max) values were 92±26.4% of that of F18-FDG SUV (max) for the primary lesions. Cu-64-TP3805 SUV (max) values for the malignant lymph nodes were 89.8±27% of those for F-18-FDG.

The whole body images revealed liver uptake of Cu-64-TP3805. This was not quantified. Although the exact nature of this uptake is unknown, preclinical data indicated that the liver uptake was 25.4±1.74%, of which nearly 60% of the activity had the same molecular weight as TP3805. Preclinical data also showed 7.5% of the activity was excreted in feces within 24 hours post injection (Thakur, et al. (2010) supra).

For the thirteen PEM patients, there were fourteen primary lesions (two in one patient) all of which were unequivocally delineated by Cu-64-TP3805. The tumor volume as determined by F-18-FDG scan ranged from 141 mm³ to 3818 mm³. The tumor volume range, as calculated from the Cu-64-TP3805, was 98 mm³ to 6912 mm³. This was 113±37% of the F-18-FDG values.

In PEM imaging, the tumor uptake values were determined as ratios of PEM uptake value (PUV) to PEM background value. These ratios for F-18-FDG ranged from 2.6 to 11.6 and those for Cu-64-TP3805 from 2.7 to 11.8. This is 97.7±24.5% of the F-18-FDG values.

These data establish the utility of the Cu-64-TP3805 probe for clinical applications in patients with BC. The Cu-64-TP3805 tumor uptake as observed in PEM imaging was rapid. The PUV values calculated from the 15-minute PEM image remained steady compared to the last image at 4 hours to 5 hours post-injection. By design, the Cu-64-TP3805 receivdd by all nineteen (six plus thirteen) patients ranged from 107.3 MBq to 162.8 MBq. Given that the positron emission of Cu-64 is only 19%, compared to 97% for F-18-FDG, the effective Cu-64-TP3805 dose ranged between 21 MBq to 33 MBq, less than one tenth of that of the F-18-FDG. Despite this small amount of the tracer, the Cu64-TP3805 image quality was excellent, both for PET/CT images and PEM images. All malignant lesions (n=20), including the malignant lymph nodes (n=4), were clearly delineated by the Cu-64-TP3805.

The use of F-18-FDG provided direct comparison of the usefulness of Cu-64-TP3805, qualitatively and quantitatively. Since Cu-64-TP3805 targets VPAC1 receptors, SUV or PUV were independent of the hormonal status of these BC lesions.

Chemical and Radiation Toxicity.

Cu-64 is a rapidly emerging γ+ radionuclide. It is used in humans for PET imaging (Anderson, et al. (2001) supra; Lewis, et al. (2001) supra; Pfeifer, et al. (2012) J. Nucl. Med. 53(8):1207-1215). Cu-64 has a t½ of 12.8 hours, which is long enough to be dispatched throughout the country, but not too long to deliver excessive radiation dose to patients after imaging. Cu-64 is produced in large quantities on small cyclotrons and its chemistry is well known. In PEM studies herein, 13 patients received 3.87±0.2% mCi and received an estimated dose of 2.52 mSv to the whole body and 36.5 mSv to the liver (target organ). Table 3 below shows data for other breast cancer (BC) imaging agents, Cu-64-TP3805, ACRIN study (ACRIN 6682, Phase II Trial 2012) and Ga-67 used in humans since 1970. Data demonstrate that Cu-64-TP3805 radiotoxicity is less than induced by the well-established radio-tracers and ACRIN-promoted Cu-64-ATSM.

TABLE 3

| | Effective | Liver | |
|---|---|---|---|
| Procedure | Dose | Dose | Reference |
| Cu-64-TP3805 | 2.5 mSv | 36.5 mSv | Gingold, eIND 101550 |
| (4 mCi) | | | |
| FDG-PET/CT | 31.9 mSv | — | Huang, et al. (2009) |
| (F-18 10 | | | Radiology 251(1): 166-174 |
| mCi) | | | |
| Sestamibi | 9.4 mSv | — | Hendrick (2010) Radiology |
| Tc-99m (30 | | | 257(1): 246-253 |
| mCi) | | | |
| FDG-PET/PM | 2.8 mSv | — | Hendrick (2010) supra |
| w/o CT (F-18 | | | |
| 10 mCi) | | | |
| Ga-67 | 39 mSv | 69 mSv | MIRD-dose estimate report |
| Citrate (10 | | | no. 2. (1973) J. Nucl. |
| mCi) | | | Med. 14(10): 755-756 |
| Cu-64 ATSM | 33.3 mSv | 361 mSv | Lewis, et al. (2008) J. |
| (25 mCi) | | | Nucl. Med. 49(7): 1177- |
| | | | 1182 |

It has been demonstrated that metal ions in >6 times the Cu-64 dose used herein are not toxic (Lewis, et al. (2008) supra). In addition, data in rabbits receiving a 1,000× dose (adjusted to body weight) of decayed Cu64-TP3805 did not exhibit elevated c-AMP, altered blood chemistry or changes in liver enzymes. No toxicity was observed in any of the 19 patients who received Cu-64-TP3805.

### Example 3

### Preparation of Lyophilized Kit

Reagents.

Deoxygenated and sterile H₂O was prepared by placing 400 ml-deionized H₂O into a 500 ml beaker and flushing the water with 0.2-micron filtered nitrogen for 5 minutes. The water was subsequently transferred to a clean, sterile 500 ml glass bottle, sealed and autoclaved. The water was cooled to room temperature and stored in at 4° C.

Deoxygenated 0.1 N HCl (used for dilution of ⁶⁴Cu) was prepared by mixing 90 ml deoxygenated H₂O with 10 ml of 1 N HCl and flushing the mixture with 0.2 micron filtered nitrogen for 60 minutes.

Deoxygenated 0.9% NaCl (used for trace labeling to make up volume) was prepared by mixing 100 ml deoxygenated H₂O with 900 mg NaCl and flushing the solution with 0.2 micron filtered nitrogen for 60 minutes.

NaOH (1.0 N) was prepared by mixing 9 ml deoxygenated H₂O with 1 ml of 10 N NaOH.

Acetate buffer (0.2 M, pH 4.6) was prepared by mixing 25.5 ml of a 0.2 M solution of acetic acid (1.155 ml in 100 ml deoxygenated H₂O) with 24.5 mL of a 0.2 M solution of sodium acetate (2.72 g of C₂H₃O₂Na.3H₂O in 100 mL of deoxygenated H₂O), diluting the mixture to a total of 100 ml using deoxygenated H₂O and flushing the solution with 0.2 micron filtered nitrogen for 60 minutes.

Tin II chloride.2H₂O (98%) was ACS grade, Glycine (98.5⁺%) was ACS grade, and Gluconic acid (sodium salt, 99%) was ACS grade.

All glassware was washed, rinsed with deionized water and baked in oven at 100° C. for 2 hours and subsequently sterilized by autoclave. Glycine (1.5014 grams) was added to 100 ml deoxy water and mixed to dissolve. The glycine solution was transferred to a 150 ml glass beaker and pH was adjusted to 9.0 with 1 N NaOH (˜1.5 ml) to make 0.2 M pH 9.0 glycine buffer. The glycine buffer was flushed with 0.2 micron filtered nitrogen for 20 minutes, and filtered with a 0.2 micron filter. A 200 μl aliquot was transferred to a vial.

To 1 ml deoxy water was added 50 mg glucoheptone. The solution was flushed with 0.2 micron filtered nitrogen for 5 minutes. To 400 μl glucoheptone solution was add 4 mg tin II chloride.2H₂O in 400 μl 0.05 M hydrochloric acid. The mixture was filtered with 0.2 micron filter, and a 40 μl aliquot was transferred to the vial containing the glycine buffer.

To 1 ml sterile 0.2 M pH 4.6 acetate buffer was add 5 mg peptide (TP3805) to make a concentration of 5 mg/ml. From this solution, 4 μl (20 μg) peptide (TP3805) was transferred to the vial containing the glycine buffer, tin chloride and acetate buffer.

The solution in the vial was frozen quickly on dry ice and subsequently dried with a lyophilizer (˜6-8 hours). At the end, nitrogen was flushed into the chamber of lyophilizer. The vial was removed, capped and stored at −20° C.

### Example 4

### Radiolabeling of Peptide

Using aseptic technique, 200 μl sterile, pyrogen-free, deoxy water (containing no preservatives) was injected into ⁶⁴Cu solution. The solution was mixed and transferred into the vial containing the peptide. The vial was shaken to ensure complete dissolution of the powder. The solution was then incubated at 50° C. for 90 minutes and subsequently allowed to stand at room temperature prior to use. In certain embodiments, the solution can be incubated at a temperature ranging from room temperature to 90° C.

1-12. (canceled)

13. A kit comprising a lyophilized composition including glucoheptonate, a buffer, and a compound having the structure His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-γAba-Lys(R) (SEQ ID NO:1), wherein R is a chelator coupled to the γ-amino group of the lysine residue.

14. The kit of claim 13, wherein the kit is at 4° C. or less.

15. The kit of claim 13, wherein the chelator comprises

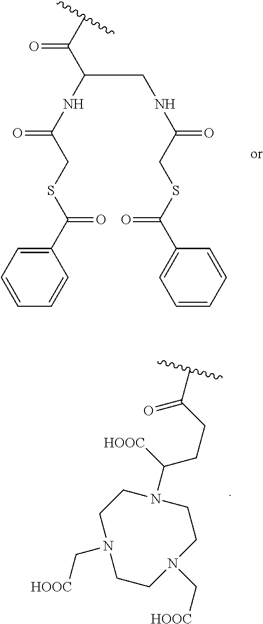 [SMILES: *C(=O)C(CNC(=O)CSC(=O)c1ccccc1)NC(=O)CSC(=O)c1ccccc1 ; *C(=O)CCC(C(=O)O)N1CCN(CC(=O)O)CCN(CC(=O)O)CC1]

16. The kit of claim 13, wherein the kit comprises at least one of the following:
(i) 50 μg to 500 μg of glucoheptonate;
(ii) glycine buffer as the buffer;
(iii) SnCl₂.2H₂O in the lyophilized composition;
(iv) a radioprotectant;
(v) antimicrobial preservative;
(vi) pH-adjusting agent;
(vii) the compound adhered, affixed or immobilized to a substrate; or
(viii) 20 μg to 250 μg of the compound.

17. The kit of claim 13, wherein the kit comprises a radionuclide separate from the lyophilized composition.

18. The kit of claim 17, wherein the radionuclide is ⁶⁴Cu, ⁶⁸Ga, ⁸⁹Zr or ^{99m}Tc.

19. The kit of claim 13, further comprising instructions for one or more of:
(a) reconstituting the lyophilized composition;
(b) radiolabeling the compound with a radionuclide;
(c) administering the composition to a subject; or
(d) interpreting results of using the kit.

20. The kit of claim 19, further comprising photos of labeling tissues with the compound, the tissues being with and without cancer.

21. A method for preparing a kit for tumor imaging comprising
(a) combining, in a container, glucoheptonate, buffer, and a compound having the structure His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-γAba-Lys(R) (SEQ ID NO:1), wherein R is a chelator coupled to the γ-amino group of the lysine residue;
(b) freezing the combination of (a),
(c) lyophilizing the frozen combination,
(d) introducing sterile nitrogen gas into the container, and
(e) sealing the container.

22. The method of claim 21, comprising storing the container is at 4° C. or less.

23. The method of claim 21, wherein the chelator comprises

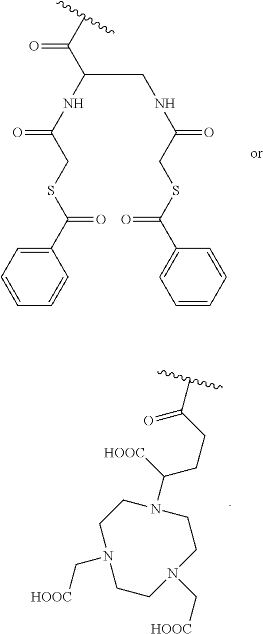 [SMILES: *C(=O)C(CNC(=O)CSC(=O)c1ccccc1)NC(=O)CSC(=O)c1ccccc1 ; *C(=O)CCC(C(=O)O)N1CCN(CC(=O)O)CCN(CC(=O)O)CC1]

24. The method of claim 21, wherein the combination of (a) comprises at least one of the following:
(i) 50 μg to 500 μg of glucoheptonate;
(ii) glycine buffer as the buffer;
(iii) SnCl₂.2H₂O;
(iv) a radioprotectant;
(v) antimicrobial preservative;
(vi) pH-adjusting agent;
(vii) the compound adhered, affixed or immobilized to a substrate; or
(viii) 20 μg to 250 μg of the compound.

25. The method of claim 21, further comprising (f) providing a radionuclide separate from the compound.

26. A method of labeling a compound of SEQ ID NO:1 comprising:
(a) providing the kit of claim 13;
(b) providing a radionuclide in solution;
(c) introducing the radionuclide to the compound;
(d) shaking the radionuclide and compound for dissolution; and
(e) incubating the radionuclide and compound at room temperature.

27. A kit comprising a compound having the structure His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-γAba-Lys(R) (SEQ ID NO:1), wherein R is a chelator coupled to the γ-amino group of the lysine residue wherein said compound is adhered, affixed or immobilized on a substrate.


SEQUENCE LISTING (1 sequence)

SEQ ID NO: 1 - protein, 29 aa, Artificial sequence
  Synthetic peptide
HSDGIFTDSYSRYRKQMAVKKYLAAVLEK